(19) United States
(12) Patent Application Publication
(10) Pub. No.: US 2004/0122408 A1
(43) Pub. Date: Jun. 24, 2004

(54) Dry-blend elastomer for elastic laminates

(76) Inventors: Prasad S. Potnis, Duluth, GA (US); David M. Matela, Alpharetta, GA (US); Sjon-Paul L. Conyer, Westmoreland, TN (US); Gregory T. Sudduth, Cumming, GA (US); Randall J. Palmer, Acworth, GA (US); Michael T. Morman, Alpharetta, GA (US); Nathan T. Mitchell, Hagerstown, IN (US); Steven R. Inabinet, Columbia, SC (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/328,850

(22) Filed: Dec. 24, 2002

(51) Int. Cl. A61F 13/15; D02G 3/00

(52) U.S. Cl. 604/385.24; 428/373

(57) ABSTRACT

An elastic laminate having at least one layer of non-elastic material and an elastic layer including a dry-blended elastomer made from a blend of olefinic plastomers and elastomeric styrenic block copolymers and essentially free of tackifiers and fillers yields an inexpensive elastomeric blend material with adequate elastic properties for use in personal products while permitting greater flexibility and control of material to the manufacturer of thermoplastic materials for personal products.

### BACKGROUND OF THE INVENTION

Personal products including diapers and sanitary pads often are made with a top sheet material (also referred to as a cover sheet or liner), an absorbent core which is the primary liquid retention layer, and a liquid impervious back sheet, or outer layer. Some such items may also have a surge layer for fluid uptake and distribution, or other specialized layers between the top sheet and absorbent core, and additional gasketing, or containment, flaps within the product. Absorption and retention of fluid, comfort, and avoidance of leakage are the functions desired of such products. Thus, garments often include elasticized portions to create a gasket-like fit around certain openings, such as waist openings and leg openings.

Laminates made from conventional elastic filaments and elastic attachment adhesive are often used to create such elasticized portions. However, such laminates can feel rough or otherwise be uncomfortable. For example, such laminates may cause red-marking on a wearer's skin if the fit is too tight, i.e., elastic tension is too high. Some laminates may result in leakage from the garment if the fit is too loose, i.e., elastic tension is too low. Some elastics may display noticeable tension decay or may become rigid and therefore negatively affect the softness and pliability of the elastic areas of the product, thereby leading to a loss of performance or aesthetics, or both.

There has been a desire in the art to make absorbent garments, such as diapers, better fitting, i.e., more closely conform to the shape of the wearer. One technique for rendering a better fit is to have at least some of the functional layers, e.g., the top and back sheets or other areas, expandable, especially laterally or transversely, in the waist area of the garment. It is known in the art that expandability of the garment can be limited by the least expandable layer when the layers are connected in the constructed garment. Known components for limited use absorbent garments and the like include single site catalyzed polymers such as metallocene catalyzed polymers including metallocene catalyzed polyolefins, e.g., ethylene, propylene, or other olefinic molecules. Examples of such single site catalyzed polymers are available under the tradename AFFINITY from Dow Chemical Co. of Midland, Mich., or others. Styrenic block copolymer materials, based on butadiene or isoprene or their hydrogenated or partially hydrogenated versions, are also used, such as are available under the tradename KRATON from Kraton Polymers of Houston, Tex., or others.

Either of these known classes of components alone may offer challenges to the manufacture of limited use personal products. For example, the extension/retraction properties of single site catalyzed polymers such as metallocene catalyzed polymers are closer to a plastomer than an elastomer, i.e., they are extensible but without great retraction, and therefore are sometimes not adequately elastic for use in all product applications. Styrenic block copolymers, while exhibiting more nearly elastomeric extension/retraction properties than metallocene catalyzed polymers, can be expensive for incorporation into limited use personal products. A combination of the two components would be desirable, especially where the combination uses less of the costly styrenic block copolymers. Examples of such blends are disclosed in U.S. Pat. No. 5,853,881 to Estey et al. However, the blends disclosed in Estey et al. provide for a high percentage of the more expensive styrenic block copolymer.

Use of preblended polymers, such as combinations of the aforementioned plastomers and elastomers, can be problematic from a manufacturing standpoint in that the manufacturer of thermoplastic materials, such as nonwoven webs or films, that purchases the preblended polymers has little control over the formulation process. The use of the preblended polymers thus results in a limited ability by the manufacturer of thermoplastic nonwoven materials to change the composition of their thermoplastic materials. Such changes in composition may need to be made by the manufacturer of thermoplastic materials between different products or even during adjustments within the manufacturing run of a single material type. Yet, such adjustments are made difficult by the use of preblended polymers. Thus, there is a further need or desire for processes and materials giving the manufacturer of thermoplastic materials greater control over the manufacture of blended polymer materials. Further, within the context of a limited use garment manufacture utilizing elastomeric materials resulting from such blends, it is of great concern to the creation of elasticized portions of the garment that the elastic material retain suitable extension and retraction properties, i.e., elastic performance, and be economical for use in a limited use garment without sacrificing performance or aesthetics, or both, of the garment.

### SUMMARY OF THE INVENTION

Surprisingly, it has been found by the inventors that dry-blending, or dry mixing, of multiple polymers can enable a manufacturer of thermoplastic materials, e.g., a personal products manufacturer, to produce materials of lower percentage styrenic block copolymer and which maintain the performance and aesthetic characteristics of materials previously made from preblended combinations. This discovery can provide great benefit to the economy and flexibility of personal product manufacture.

In response to the discussed difficulties and problems encountered in the prior art, dry-blended elastomers and plastomers, and garments utilizing the dry-blend, are contemplated. In certain aspects of the present invention, any garment opening such as a waist opening, sleeve or leg cuffs, or necklines may benefit from being made elastic or having elastic components added thereto to improve the fit of the garment against the body, hereinafter referred to as “elasticized.” The margins of any garment opening may hereinafter be collectively referred to as “cuffs” or “cuff areas.” Certain aspects of the present invention may provide any one of an elasticized cuff area, non-cuff area, as further explained in conjunction with the detailed explanation, having extensibility and elasticity for improved fit and the reduced leakage of exudates. It is desired that personal products, e.g., absorbent articles and garments, and especially garments such as diapers, training pants or incontinence garments, provide a close, comfortable fit about the body of the wearer and contain body exudates while maintaining skin health. In certain circumstances, it is also desirable that such garments are capable of being pulled up or down over the hips of the wearer to allow the wearer or care giver to easily pull the article on and easily remove the article. Other garment openings such as sleeve or pant cuffs and necklines may benefit from being similarly elasticized, as noted above.

This invention is generally directed to dry-blends of two polymers, commonly used in personal products, to create a dry-blended elastomer with desired elastic characteristics.

Particularly, the dry-blended elastomer may be optimized at anytime during the manufacturing process for use in personal products such as absorbent articles with elastic materials, including elastomeric films or elastic filaments, to improve the elastic properties of the manufactured product. The elastomer blend can, if desired, then be made into a laminate, e.g., with nonwoven facings, for use in, or as, certain layers of a personal product.

The elastic material layer is preferably made of a blend of a conventional elastomer, and a narrow, or low, polydispersity number polyolefin plastomer, e.g., having a polydispersity of 4 or less. Polydispersity number, sometimes called polydispersity index, is defined as weight average molecular weight divided by number average molecular weight. Polymers produced using the metallocene catalyzing process have the unique advantage of having a very narrow molecular weight range. Polydispersity numbers (Mw/Mn) of below 4 and even below 2 are possible for metallocene catalyzed polymers. These polymers also have a controlled short chain branching distribution when compared to otherwise similar Ziegler-Natta catalyzed type polymers. The reader is referred to the aforementioned U.S. Pat. No. 5,853,881 to Estey et al. for further discussion.

It is also desirable that such polyolefin plastomers have a density of between about 0.80 to about 0.95 grams per cubic centimeter (g/cc) and desirably about 0.86 to about 0.90 grams per cubic centimeter in order to maintain the elastic performance necessary for commercial applications in the personal product field. One way of measuring how well elastic materials perform is by measuring their hysteresis. Hysteresis, as used herein, is a measure of how well an elastic material retains its elastic properties between extension and retraction. A material with no hysteresis would show the same force measured at, e.g., 30 percent elongation during the retraction, or second, half-cycle as the force of extension at 30 percent elongation during the elongation, or first, half-cycle. Percentage of hysteresis may be obtained by subtracting the second half-cycle force of retraction from the first half-cycle force of extension and dividing this number by the first half-cycle force of extension (both at 30 percent elongation, e.g., during a 100% extension/retraction cycle) and multiplying by 100. A material with no difference in force between the extension and retraction half-cycles would have a zero percent hysteresis. A material with some hysteresis would have a hysteresis percentage number above zero. Smaller percentage hysteresis is considered better for present purposes.

In certain aspects of the present invention expandable polyolefin plastomers, e.g., single site catalyzed polyolefins such as metallocene catalyzed polyethylene such as, e.g., commercially available under the trade names AFFINITY or ENGAGE from Dow Chemical of Midland, Mich., or other polyolefin plastomers known in the art including polypropylene based plastomers or others; and styrenic block copolymers, such as ones commercially available under the trade name KRATON G1657 from Kraton Polymers, of Houston Tex., are blended together using a dry-blend technique without sacrifice of desired elastic behavior. An elastic film material of adequate performance for use in personal products can be made from the dry-blend of the present invention with under fifty weight percent of the styrenic block copolymer. The dry-blend is desirably made substantially without fillers or tackifiers which may interfere with the elastic performance of the film.

The blend material may also be made into filaments or elastomeric webs and utilized in laminates with other filaments, webs, or films which can be incorporated into personal products to provide expandable areas such as elastomeric cuff areas or other areas for garments to improve the elastic characteristics of such areas, thereby providing good aesthetics and performance for such garments.

Aspects of the present invention are directed to garments utilizing elastic blends, and laminates incorporating such elastic blends, to provide adequate elastic properties. The elastic blend laminates utilized in certain aspects of the invention can be made up of a combination of, e.g., a nonwoven facing or facings and elastomeric filaments or films. A layer of spunbond or other facing material can be laminated along one, or both, surfaces of the film to provide the elastic blend laminates of the invention. Alternatively, it is envisioned that laminates according to the present invention may be produced utilizing the elastic filaments or films placed between primary garment layers such as the back sheet, or outer cover, and liner, or top sheet, of the garment. A combination of elastomeric filaments and films might also be suitably used.

The dry-blended elastomer may be formulated to provide a variety of materials with differing tension properties. For example the rate and extent of tension, and hysteresis characteristics between the expansion and contraction, may be readily varied according to the dictates of the elastic material's application within the product.

### BRIEF DESCRIPTION OF THE DRAWINGS

The accompanying drawings are presented as an aid to explanation and understanding of various aspects of the present invention only and are not to be taken as limiting the present invention.

FIG. 1 illustrates a first garment according to the present invention, in this case an exemplary diaper.

FIGS. 2-3 illustrate preblended polymer and dry-blended polymer processes, respectively.

FIGS. 4-6 are graphs illustrating the tension loads at thirty percent extension, thirty percent retraction, and comparative hysteresis between dry-blended and precompounded polymer blend examples, respectively.

FIG. 7 is a graph illustrating the comparative percentage of film set between dry and precompounded polymer blend examples.

FIG. 8 is a graph illustrating the comparative retained energy between dry and precompounded polymer blend examples.

FIGS. 9 and 10 are graphs illustrating the comparative hysteresis between blend examples with different types of metallocene catalyzed polymers.

FIG. 11 is a graph illustrating the comparative energy retention between dry and precompounded polymer blend examples.

FIG. 12 shows one exemplary lamination method suitable for making a laminate according to the invention.

### DEFINITIONS

Within the context of this specification, each term or phrase below will include the following meaning or meanings.

“Bonded” refers to the joining, adhering, connecting, attaching, or the like, of at least two elements. Two elements will be considered to be bonded together when they are bonded directly to one another or indirectly to one another, such as when each is directly bonded to intermediate elements.

As used herein, the term “consisting essentially of” does not exclude the presence of additional materials which do not significantly affect the desired characteristics of a given composition or product. Exemplary materials of this sort would include, without limitation, pigments, antioxidants, stabilizers, surfactants, waxes, flow promoters, solvents, particulates, and materials added to enhance processability of the composition.

“Denier” refers to a measure of the linear density of fibers in grams per 9000 meters of fiber.

“Dry-blending” refers to the mixing of materials with no substantial chemical reaction between the materials and the extruding of the unreacted mixture in one step.

“Elastic tension” refers to the amount of force per unit weight required to stretch an elastic material (or a selected zone thereof) to a given percent elongation.

“Elastomeric” and “elastic” are sometimes used interchangeably to refer to a material or composite which can be elongated by at least 50 percent of its relaxed length and which will recover with force, upon release of the deformation stress, at least 40 percent of its elongation. It is generally desirable that an elastomeric material or composite be capable of being elongated under low stress by at least 100 percent, more preferably by at least 300 percent, of its relaxed length and recover with force, upon immediate release of the deformation stress, at least 50 percent of its elongation.

An “elastomer” is an elastic polymer. A “plastomer” is an extensible polymer. Polymers which are capable of stretching several times their original dimension when a force is applied and then quickly recover or regain the original dimension or nearly the original dimension when the force is removed are known to exhibit rubber elastic behavior. Polymers which are capable of deformation under the influence of a force but have little or no tendency to regain shape upon the removal of the force are plastic. Plastomers are neither fully elastic nor plastic but show varying degree of elasticity and plasticity under given conditions. Hence, some of their properties, for instance stress-elongation, may appear to be elastic. A plastomer may show 1000%, or 800% or 600% elongation at break. However, it may give low modulus in the range of 1000 to 7000 psi in certain tests such as hysteresis and tension set, as the elongation becomes higher and higher, a plastomer will show plastic like behavior with a high percentage set and hysteresis while an elastomer in a similar condition gives a low percentage set, and hysteresis.

“Elongation”, refers to the capability of a material to be stretched a certain distance, such that greater elongation refers to a material capable of being stretched a greater distance than a material having lower elongation. “Extensibility” and “expandability” will generally be considered as having the same meaning and may refer to a material property of elongation which does not necessarily recover its shape.

“Film” refers to a thermoplastic film made using a film extrusion process, such as a cast film or blown film extrusion process. The term includes apertured films, slit films, and other porous films which constitute liquid transfer films, as well as films which do not transfer liquid.

“Garment” includes personal care garments, medical garments, and others. The term “medical garment” includes medical (i.e., protective and/or surgical) gowns, caps, gloves, drapes, face masks, and the like. The term “industrial workwear garment” includes laboratory coats, cover-alls, and the like.

The terms “limited use” and “disposable” when used in association with personal products or garments include products which are typically and economically disposed of after 1-5 uses, are economically discarded when soiled, and are not intended for reuse.

“Incorporate” and “Blend” refer to the process of combining two or more elements into a single structure intended to be inseparable.

“Layer” when used in the singular can have the dual meaning of a single element or a plurality of elements.

The term “machine direction” means the length of a fabric in the direction in which it is produced. The term “cross direction” or “cross machine direction” means the width of fabric, i.e. a direction generally perpendicular to the machine direction.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al., which is incorporated herein in its entirety by reference. Meltblown fibers are microfibers which may be continuous or discontinuous, are generally smaller than about 0.6 denier, and are generally self bonding when deposited onto a collecting surface.

As used herein, the term “neck” or “neck stretch” interchangeably means that the fabric is extended under conditions reducing its width or its transverse dimension. The controlled extension may take place under cool temperatures, room temperature or greater temperatures and is limited to an increase in overall dimension in the direction being extended up to the elongation required to break the fabric. The necking process typically involves unwinding a sheet from a supply roll and passing it through a brake nip roll assembly driven at a given linear speed. A take-up roll or nip, operating at a linear speed higher than the brake nip roll, extends the fabric and generates the tension needed to elongate and neck the fabric. U.S. Pat. No. 4,965,122, to Morman, which is incorporated herein in its entirety by reference, discloses a process for providing a reversibly necked nonwoven material which may include necking the material, then heating the necked material, followed by cooling. U.S. Pat. No. 5,336,545 to Morman, which is incorporated herein in its entirety by reference, discloses a composite elastic necked-bonded material including at least one necked material joined to at least one elastic sheet.

As used herein, the term “neckable material or layer” means any material which can be necked such as a nonwoven, woven, or knitted material. As used herein, the term “necked material” refers to any material which has been extended in at least one dimension (e.g. lengthwise), reducing the transverse dimension (e.g. width), such that when the extending force is removed, the material can be pulled back, or relaxed, to its original width. The necked material typically has a higher basis weight per unit area than the un-necked material. When the necked material returns to its original un-necked width, it should have about the same basis weight as the un-necked material. This differs from stretching/orienting a material layer, during which the layer is thinned and the basis weight is permanently reduced.

Typically, necked nonwoven fabric materials are capable of being necked from about 10 to about 80 percent, desirably from about 20 to about 60 percent, and more desirably from about 30 to about 50 percent for improved performance. For the purposes of the present disclosure, the term “percent necked” or “percent neckdown” refers to a ratio or percentage determined by measuring the difference between the pre-necked dimension and the necked dimension of a neckable material, and then dividing that difference by the pre-necked dimension of the neckable material and multiplying by 100 for percentage. The percentage of necking (percent neck) can be determined in accordance with the description in the above-mentioned U.S. Pat. No. 4,965,122.

“Nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Personal products” shall include: absorbent articles used to absorb any fluid including human body fluids, such as diapers, adult incontinence garments, training pants, absorbent swim pants, feminine care products, hygienic wipes, absorbent pads and the like; disposable tissue products for personal use, such as bath tissue, facial tissue, paper towels and napkins; disposable apparel for institutional, industrial and consumer use; disposable health care products that are not intended to be cleaned for reuse, such as caps, gowns, foot wear, masks, drapes, wraps, covers, and the like; consumer health care products; and health care or environmental diagnostic devices that are at least partially disposable.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Spunbond fiber” refers to small diameter fibers which are formed by extruding molten thermoplastic material as filaments from a plurality of fine capillaries of a spinneret having a circular or other configuration, with the diameter of the extruded filaments then being rapidly reduced as taught, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Thermoplastic” describes a material that softens when exposed to heat and which substantially returns to a nonsoftened condition when cooled to room temperature.

“Vertical filament stretch-bonded lamination” or “VFSBL” refers to a stretch-bonded lamination process using a continuous vertical filament process.

Words of degree, such as “about”, “substantially”, and the like are used herein in the sense of “at, or nearly at, when given the manufacturing, design, and material tolerances inherent in the stated circumstances” and are used to prevent the unscrupulous infringer from unfairly taking advantage of the invention disclosure where exact or absolute figures are stated as an aid to understanding the invention.

These terms may be defined with additional language in the remaining portions of the specification.

### DETAILED DESCRIPTION

The various aspects and embodiments of the invention may be described in the context of disposable absorbent articles, and more particularly referred to, without limitation and by way of illustration only, as a disposable diaper. It is, however, readily apparent that the present invention could also be employed to produce other products or garments, such as feminine care articles, various incontinence garments, medical garments and any other disposable garments. Typically, the disposable garments are intended for limited use and are not intended to be laundered or otherwise cleaned for reuse. A disposable diaper, for example, is economically discarded after it has become soiled by the wearer.

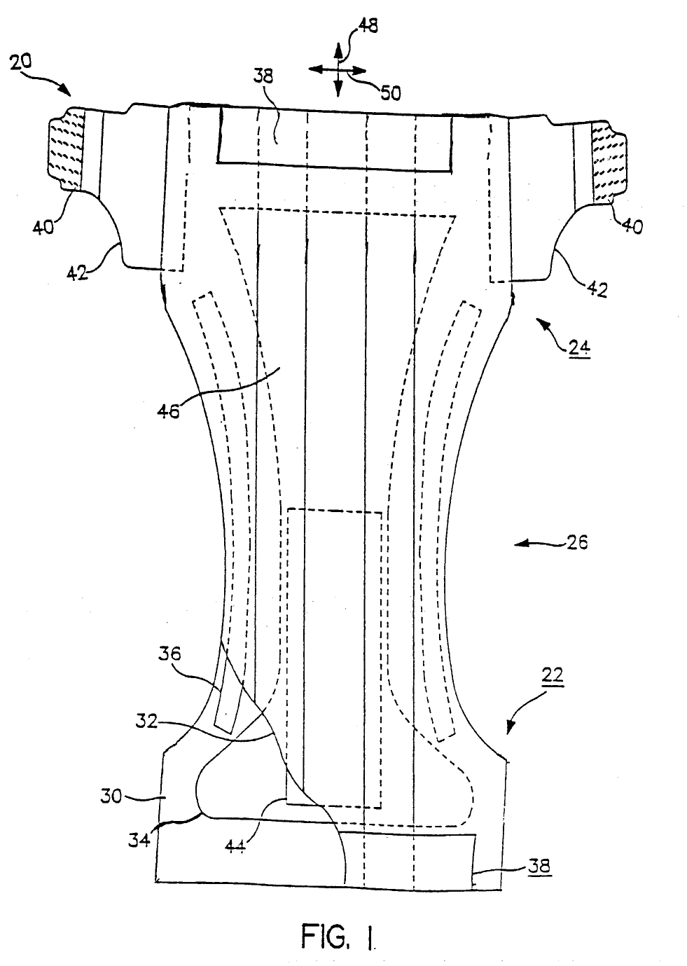

FIG. 1 is a representative plan view of a personal product, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs.

The diaper 20 includes, without limitation, an outer cover, or back sheet 30, a liquid permeable bodyside liner, or top sheet, 32 positioned in facing relation with the back sheet 30, and an absorbent core, or body, being the primary liquid retention structure 34, such as an absorbent pad, which is located between the back sheet 30 and the top sheet 32. The back sheet 30 defines a length, or longitudinal direction 48, and a width, or lateral direction 50 which, in the illustrated embodiment, coincide with the length and width of the diaper 20. The liquid retention structure 34 generally has a length and width that are less than the length and width of the back sheet 30, respectively. Thus, marginal portions of the diaper 20, such as marginal sections of the back sheet 30, may extend past the terminal edges of the liquid retention structure 34. In the illustrated embodiment, for example, the back sheet 30 extends outwardly beyond the terminal marginal edges of the liquid retention structure 34 to form side margins and end margins of the diaper 20. The top sheet 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer. The person having ordinary skill in the art will appreciate that other areas, such as the front waist section 22, or the entire area of the diaper 20 such as covered by top sheet 32, may be made expandable. Any expandable areas of the diaper 20 may utilize the elastics or laminates as taught herein.

In the illustrated embodiment, the diaper 20 includes a pair of side panels 42 to which fasteners 40, indicated as the hook portion of a hook and loop fastener, are attached. Generally, the side panels 42 are attached to the side edges of the diaper 20 in one of the waist sections 22, 24 and extend laterally outward therefrom. The side panels 42 may be expandable. For example, the side panels 42, or indeed, any precursor component webs of the garment, may be a laminate as taught herein and may utilize processes known in the art such as a neck-bonded laminate (NBL) or stretch-bonded laminate, (SBL) process. Methods of making such materials are well known to those skilled in the art and are described in U.S. Pat. No. 4,663,220 to Wisneski et al., U.S. Pat. No. 5,226,992 to Morman, and European Patent Application No. EP 0 217 032 published on Apr. 8, 1987 in the names of Taylor et al., each of which is incorporated herein in its entirety by reference. Examples of absorbent articles that include elasticized side panels and selectively configured fastener tabs are described in PCT Patent Application No. WO 95/16425 published Jun. 22, 1995 in the name of Roessler; U.S. Pat. No. 5,399,219 to Roessler et al.; U.S. Pat. No. 5,540,796 to Fries; U.S. Pat. No. 5,595,618 to Fries and U.S. Pat. No. 5,496,298 to Kuepper et al., each of which is incorporated herein in its entirety by reference.

The diaper 20 may also include a surge management layer 44, located between the top sheet 32 and the liquid retention structure 34, to rapidly accept fluid exudates and distribute the fluid exudates to the liquid retention structure 34 within the diaper 20. The diaper 20 may further include a ventilation layer (not illustrated) located between the liquid retention structure 34 and the back sheet 30 to insulate the back sheet 30 from the liquid retention structure 34 to reduce the dampness of the garment at the exterior surface of the back sheet 30. Examples of suitable surge management layers 44 are described in U.S. Pat. No. 5,486,166 to Bishop; U.S. Pat. No. 5,490,846 to Ellis; U.S. Pat. No. 5,364,382 to Latimer et al.; U.S. Pat. No. 5,429,629 to Latimer et. al., and U.S. Pat. No. 5,820,973 to Dodge, II, et al., each of which is incorporated herein in its entirety by reference.

The disposable diaper 20 may also include a pair of expandable containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

The present invention incorporates elastic blend materials such as filaments, films, or webs, and elastic blend laminates having adequate elastic properties. The blend materials and laminates can be incorporated into any suitable article, such as personal care garments, medical garments, and industrial workwear garments. More particularly, the elastic blends and elastic blend material laminates are suitable for use in diapers, training pants, swim wear, absorbent underpants, adult incontinence products, feminine hygiene products, protective medical gowns, surgical medical gowns, caps, gloves, drapes, face masks, laboratory coats, and coveralls.

A number of elastomeric components are known for use in the design and manufacture of such articles. For example, disposable absorbent garments are known to contain expandable and elasticized leg cuffs, elasticized waist portions including cuff areas thereof, elasticized side panels and fastening tabs. The elastic blend materials and elastic blend material laminates of this invention may be applied to any suitable article to form such expandable and elasticized areas.

Suitable blends from which the elastomeric films, fibers and webs may be made include plastomer and elastomer polymers, including sufficient amounts of an elastomeric styrenic block co-polymer and a polyolefinic plastomer. The elastomeric blend may desirably include about 30% to about 40% by weight of a styrenic block co-polymer and about 70% to about 60% by weight of a metallocene catalyzed polyolefin.

Thus the present invention results in an economical material using substantially less of the comparatively expensive diblock, triblock, tetrablock, or other multi-block styrenic block copolymers; including styrene-isoprene-styrene, styrene-butadiene-styrene, styrene-ethylene/butylene-styrene, or styrene-ethylene/propylene-styrene, or styrene-ethylene/propylene-ethylene/propylene-styrene. One suitable elastic polymer may be obtained from Kraton Polymers, Inc., under the trade designation KRATON G1657. The material will use a greater weight percentage of less expensive metallocene-catalyzed polyolefins available under the tradenames AFFINITY XUS58380.01L or AFFINITY EG8200 from Dow Chemical Company, as referenced above. However, it has been found that the blend formulations of the present invention should be essentially free of tackifiers and fillers in order to maintain the desired elastic performance.

It is also possible to have other materials blended in minor amounts with the polymers according to this invention like fluorocarbon chemicals to enhance chemical repellence which may be, for example, any of those taught in U.S. Pat. No. 5,178,931, to Perkins et al., herein incorporated by reference in its entirely, fire retardants, ultraviolet radiation resistance improving chemicals and pigments to provide color for the materials. Fire retardants and pigments for spunbond and meltblown thermoplastic polymers are known in the art and are internal additives. A pigment, e.g. TiO₂, if used, is generally present in an amount less than 5 weight percentage of the layer while other materials may be present in a cumulative amount less than 25 weight percent. These additives are not considered tackifiers or fillers within the meaning of the present invention. Fabrics according to some aspects of this invention may also have topical treatments applied to them for more specialized functions. Such topical treatments and their methods of application are known in the art and include, for example, alcohol repellence treatments, anti-static treatments and the like, applied by spraying, dipping, etc.

The elastic material layer is preferably made of a blend of a conventional elastomer, and a narrow, or low, polydispersity number polyolefin plastomer, e.g., having a polydispersity of 4 or less. The desired polydispersity range will be dependent somewhat upon the final form of the elastic blend desired, e.g., film or filament, and the necessary control of the rheological properties and processability of the material, but will generally be within the range of 1 to 4 to be considered “low”.

It is also desirable that the polyolefin plastomer have a density of between about 0.80 to about 0.95 grams per cubic centimeter, desirably about 0.86 to about 0.90 grams per cubic centimeter, and desirably less than 0.90 grams per cubic centimeter, when using metallocene catalyzed polyolefins in order to maintain the elastic performance necessary for commercial applications in the personal product field.

A film of the present invention is generally a dry-blend processed mixture of the block copolymer such as a styrenic block copolymer and a single site catalyzed polymer, such as a metallocene catalyzed polyolefin polymer, and, if used, any additional components. The film may be made in a dry-blend method substantially in accordance with U.S. Pat. No. 6,261,278 to Chen et al., which is hereby incorporated by reference in its entirety.

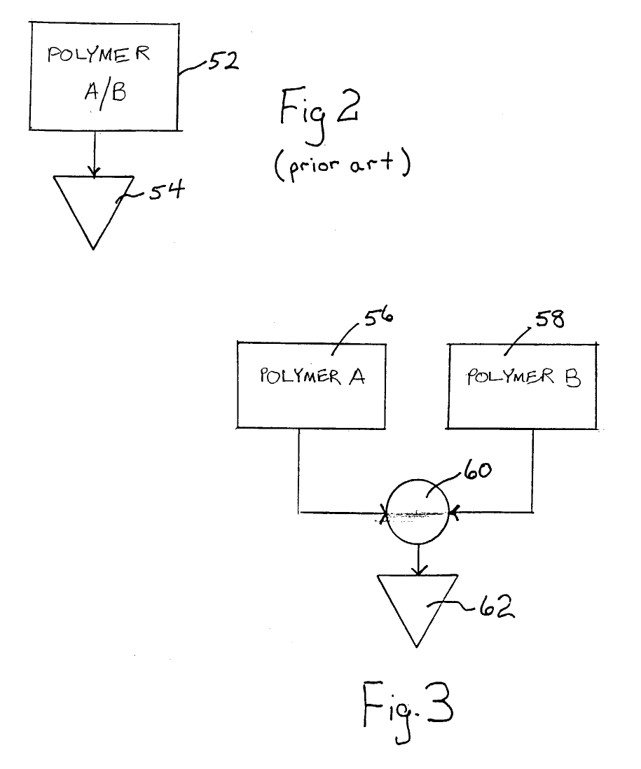

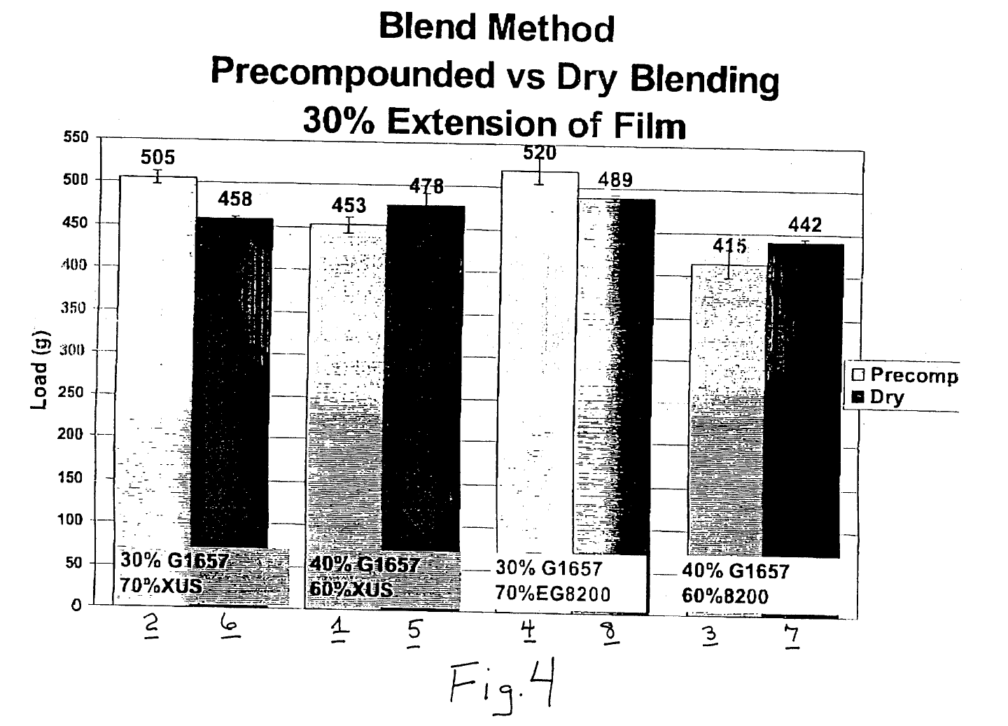

Referencing FIG. 2, according to the known art, a precompounded polymer mix 52 is fed directly to an extruder 54 which produces the thermoplastic melt to produce a film, ribbons, filaments or the like. Referencing FIG. 3, the process of dry-blending according to the present invention shows a first polymer bin 56 and a second polymer bin 58, respectively, which feed into a blender 60. The blender 60 then feeds into an extruder 62 which produces the thermoplastic melt to produce a film, ribbons, filaments or the like according to the present invention. Although shown as two components, the blender 60 and extruder 62 may be housed as a common unit. It is notable that precompounded polymer mixes are generally made with a twin screw mixer while the dry-blend of the present invention can be made with a single screw mixer. The single screw mixer is generally a less expensive piece of equipment which may allow faster throughput, i.e. mixing rates, for the manufacturer of thermoplastic materials. In one embodiment of the present invention, after dry mixing together the block copolymer and the single site catalyzed polymer to form a dry mixture, such dry mixture is beneficially agitated, stirred, or otherwise blended to effectively uniformly mix the components such that an essentially homogeneous dry mixture is formed. The dry mixture may then be melt blended in, for example, an extruder to effectively uniformly mix the components such that an essentially homogeneous melted mixture is formed. The essentially homogeneous melted mixture may then be used directly, e.g., may be formed into a film or sent directly to other equipment for forming films, thereby avoiding thermal degradation of the polymers which may lessen the elastic performance through repeated melt histories of the blend. Alternative methods of mixing together the components of the present invention include first adding the block copolymer to an extruder and then adding the single site catalyzed polymer to such an extruder, wherein the components being used are effectively mixed together within the extruder. In addition, it is also possible to initially melt mix both of the components together at the same time. Other methods of mixing together the components of the present invention are also possible and may be recognized by one skilled in the art.

The process of cooling the extruded thermoplastic composition, in the form of a film, ribbons, or filaments, to ambient temperature is usually achieved by letting the extruded film cool as is or by blowing ambient or sub-ambient temperature air over the extruded film, or extruding onto a chill roll or other controlled temperature roll.

It is generally desired that the melting or softening temperature of a thermoplastic composition comprising the block copolymer and the single site catalyzed polymer be within a range that is typically encountered in most process applications. As such, it is generally desired that the melting or softening temperature of the thermoplastic composition beneficially be between about 25° C. to about 350° C., more beneficially be between about 50° C. to about 300° C., and suitably be between about 60° C. to about 200° C.

It is generally desired that each of the block copolymer and the single site catalyzed polymer be melt processable. It is therefore desired that the block copolymer and the single site catalyzed polymer used in the present invention each exhibit a melt flow rate that is beneficially between about 1 gram per 10 minutes to about 600 grams per 10 minutes, suitably between about 5 grams per 10 minutes to about 200 grams per 10 minutes, and more suitably between about 10 grams per 10 minutes to about 150 grams per 10 minutes. The melt flow rate of a material may be determined according to a test procedure such as ASTM Test Method D1238-E.

Typical conditions for thermally processing a thermoplastic composition include using a shear rate that is beneficially between about 100 seconds⁻¹ to about 5000 seconds⁻¹ more beneficially between about 500 seconds⁻¹ to about 5000 seconds⁻¹ suitably between about 1000 seconds⁻¹ to about 3000 seconds⁻¹ and most suitably at about 1000 seconds⁻¹. Typical conditions for thermally processing the components also include using a temperature that is beneficially between about 100° C. to about 500° C., more beneficially between about 150° C. to about 300° C., suitably between about 175° C. to about 250° C., and suitably about 200° C. The film of the present invention may generally be of any size or dimension as long as the film exhibits the desired properties as described herein.

The facing sheets to be applied to the dry blend materials may be extensible or non-extensible depending upon their ultimate application in the product. In one aspect of the invention, the facing sheets could be necked, or gathered, in order to allow them to be stretched after application of the elastic blend materials. Facing layers including a known necked nonwoven facing layer such as 0.2-2.0 osy spunbond may be bonded to the elastomeric blend materials by adhesives, thermal bonding, ultrasonic bonding or other known methods.

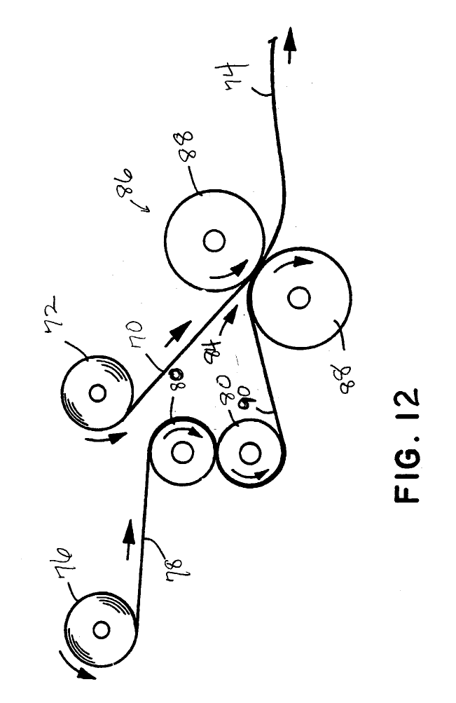

Referencing FIG. 12, a process for making one exemplary laminate 74 according to the present invention is shown. The elastomeric blend in the form of a film 70 is unwound from a supply roll 72. The film suitably has a thickness of about 0.001 inch (0.025 mm) to about 0.05 inch (1.27 mm), alternatively of from about 0.001 inch (0.025 mm) to about 0.01 inch (0.25 mm), and a width of from about 0.05 inch (1.27 mm) to about 3.0 inches (7.62 cm), alternatively of from about 0.5 inch (1.27 cm) to about 15 inches (38.1 cm). The elastomeric film 70 may also be capable of imparting barrier properties in an application.

In order to form the elastic laminate 74, at least one, or a first, roll 76, respectively, of spunbond facing material 78, such as a 0.2 to 2.0 spunbond nonwoven having fiber denier of approximately 2.0-2.5, and e.g., containing a web of spunbond polypropylene filaments, or a web of filaments of approximately 50% Polyethylene and 50% Polypropylene in a side-by-side configuration, and thermally point bonded, is fed between tensioning S-rollers, collectively 80 to be initially necked.

The elastic blend material 70 passes through the nip 84 of the bonder roller arrangement 86 formed by the bonder rollers 88 collectively. The initially necked spunbond material 78 then passes through the nip 84 of the bonder roller arrangement 86. Because the peripheral linear speed of the S-rollers 80 is controlled to be less than the peripheral linear speed of the rollers 88 of the bonder roller arrangement 86, the initially necked spunbond material 78 is further tensioned, as at 90, between the S-rollers 80 and the nip 84 of the bonder roll arrangement 86. By adjusting the difference in the speeds of the rollers, the spunbond facing material is tensioned so that it necks a desired amount, e.g. 50%, and is maintained in such tensioned, necked condition while the elastic film material 70 is joined to the necked spunbond material during their passage through the bonder roller arrangement 86 to form a composite elastic necked-bonded laminate 74.

The material layers may be bonded by an adhesive (not shown) such as Findley adhesive 2525A, from Bostik Findley Adhesives of Wauwatosa, Wis., or other suitable adhesives. The spunbond facing material 78 might also be made in situ rather than unrolled from previously-made rolls of material. While illustrated as having only one lightweight necked spunbond facings, it will be appreciated that two facing materials, or various types of facing materials, may be used. It will be appreciated that other processes and forms of the materials consistent with the present invention may be used such as the aforementioned SBL process, a horizontal lamination process as taught in U.S. Pat. No. 5,385,775 to Wright, or a vertical filament lamination process (VFL) as taught in published US Patent Application No. US2002-0104608, both of which references are hereby incorporated by reference in their entirety; or combinations of known lamination processes.

The below dry-blended film Examples 5-8 include a blend of styrenic block copolymer/mPE (metallocene catalyzed Polyethylene) made according to the present invention. Precompounded Examples 1-4 include a blend of styrenic block copolymer/mPE (metallocene catalyzed Polyethylene) made by a preblend manufacturer, such as Standridge Color Corporation, of Social Circle, Ga. The styrenic block copolymer is KRATON G1657, while the mPE (metallocene catalyzed Polyethylene) is one of trade name XUS58380.01L (hereinafter “XUS”) or trade name EG8200 (hereinafter “8200”). The blend components were blended at one of either a 30/70 or a 40/60 KRATON/mPE weight percent ratio, as indicated.

| | | |
|---|---|---|
| Example 1 | G1657/XUS 40/60 | precompounded |
| Example 2 | G1657/XUS 30/70 | precompounded |
| Example 3 | G1657/8200 40/60 | precompounded |
| Example 4 | G1657/8200 30/70 | precompounded |
| Example 5 | G1657/XUS 40/60 | dry-blend |
| Example 6 | G1657/XUS 30/70 | dry-blend |
| Example 7 | G1657/8200 40/60 | dry-blend |
| Example 8 | G1657/8200 30/70 | dry-blend |

The polymer compounds of the above examples were generally formed into films at a melt temperature of about 350-450° F., with a small single stage extruder with a casting speed of about 200-300 feet per minute to yield a finished film width of about 10 inches and a basis weight of between about 28-45 grams per square meter (gsm).

Exemplary test laminates were constructed as neck-bonded laminates (NBL)by casting an elastomeric film, according to each of Examples 1-8, between two 0.75 osy-0.85 osy polypropylene spunbond (SB) layers of about 128″ necked to 56% necking, and accordingly sized to the elastomeric film and bonding the layers with an adhesive spray of Findley adhesive 2525A to give a cross machine direction (CD) stretch material as may be useful for diaper side panels. All comparative samples of the film and laminates were made with equal processing parameters.

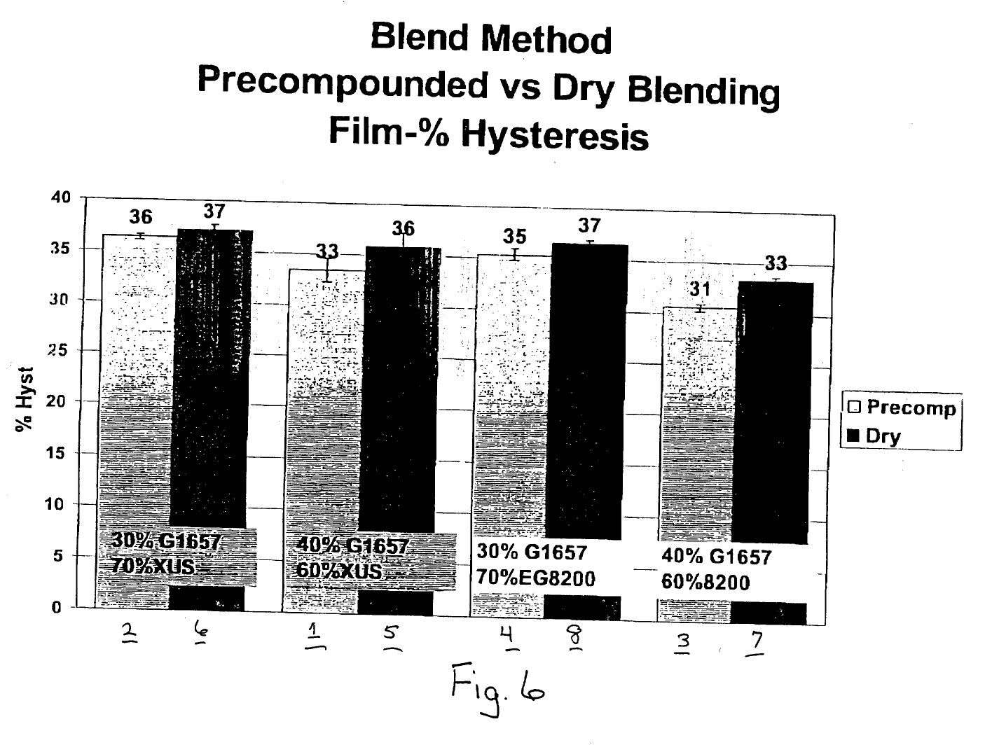

Referencing FIG. 5, a comparison graph of tension load on film samples at a 30% retraction of the films is shown. Results are labeled by the Example numbers above. It is apparent from the graph that insignificant differences in retraction tension load exist between dry-blended and precompounded examples of the polymer blend. The percentage of hysteresis results as illustrated in FIG. 6 also reveal that hysteresis differences are insignificant between the dry-blended and precompounded polymer blends of similar composition.

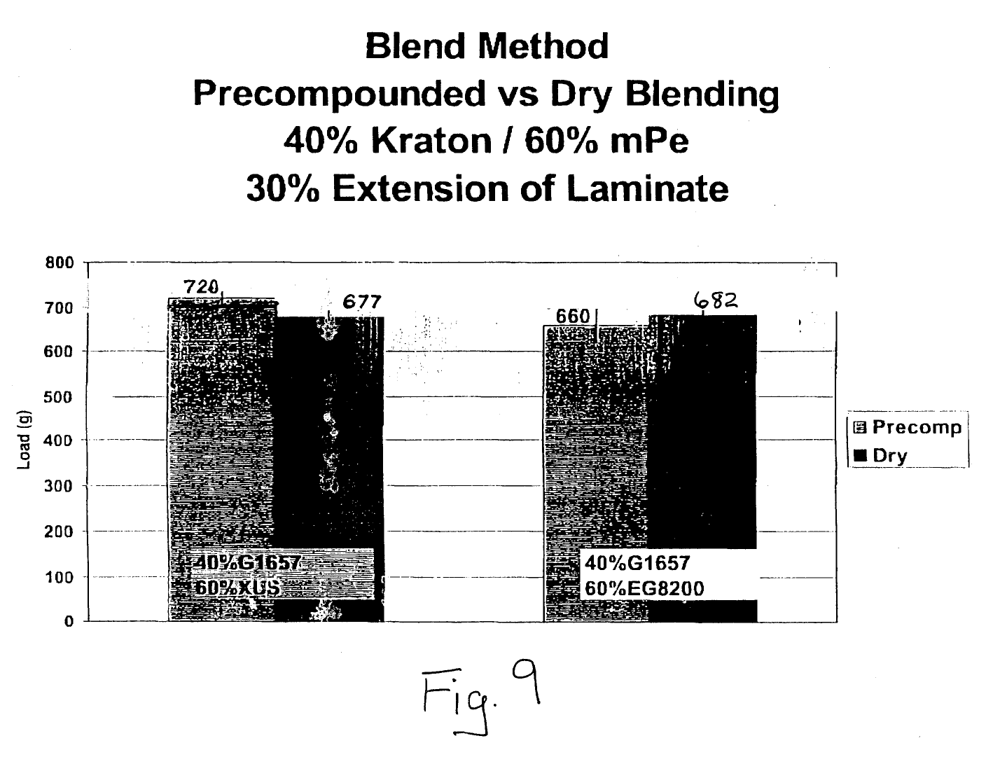

As seen in FIG. 8, a graph comparing energy retention [energy retained=1−((30% Extension−30% Retraction)/30% Extension)] of the exemplary films after a one cycle extension/retraction test, the energy retention of the composition having a higher percentage of styrenic block copolymer was higher, as might be expected. Energy retention levels can have an effect on the perceived performance of elastic side panels and fastening elements for consumer preference. The data of FIG. 8 reveals that energy retention differences are insignificant between the dry-blended and precompounded polymer blends of similar composition.

From the foregoing examples it can be seen that no significant differences in elastic performance regarding elongation tension, retraction tension, or hysteresis were obtained for the elastic performance between dry-blend material and precompounded blend material when making comparable films. Thus there is little elastic performance lost and much manufacturing efficiency to be gained from utilization of dry-blend processing according to the present invention.

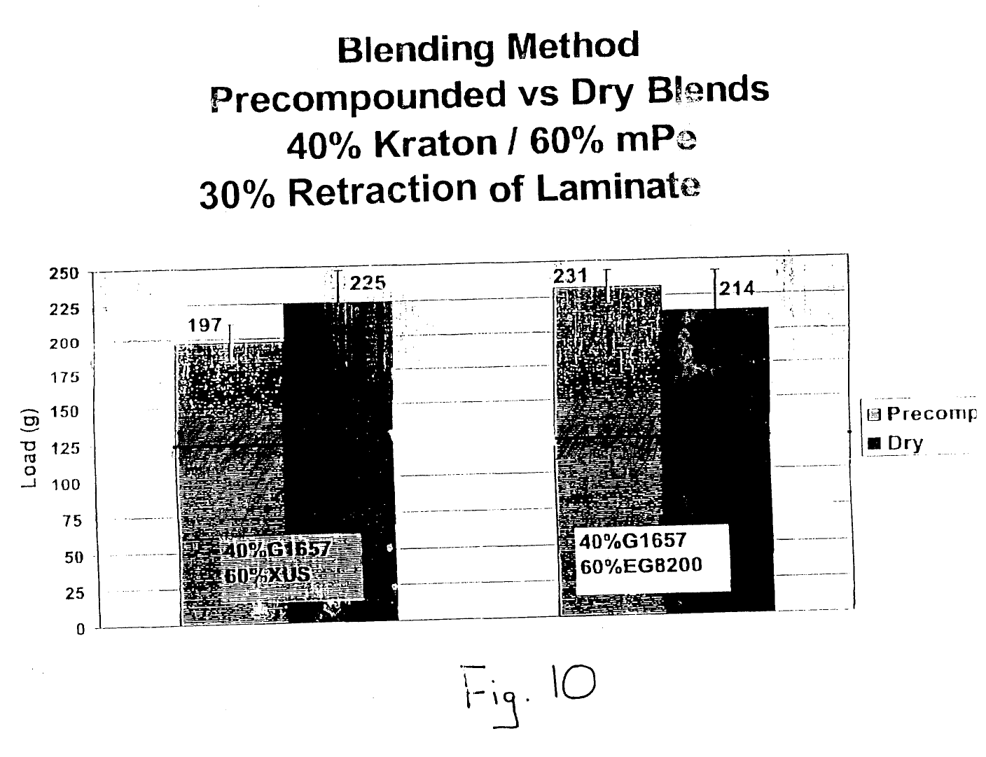

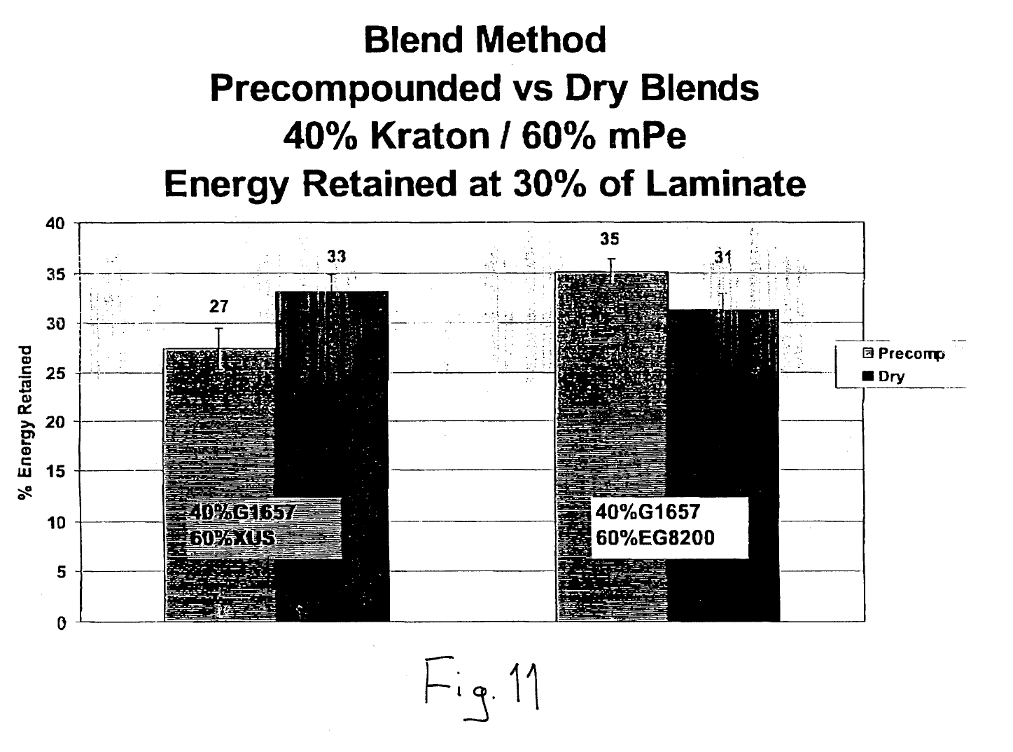

As seen in FIGS. 9-10, a comparison of expandable laminates suitable for a garment or absorbent article, as made from the above detailed film examples and facings, shows no significant differences in hysteresis performance between dry-blend material and precompounded blend material when used in the laminates. As seen in FIG. 11, a comparison of expandable laminates with 40%/60% Kraton/mPE ratio formulations shows no significant differences in energy retention performance after a one hundred percent extension/retraction cycle, at 30 percent of each half-cycle, between the dry-blend material and precompounded blend material of either test formulation.

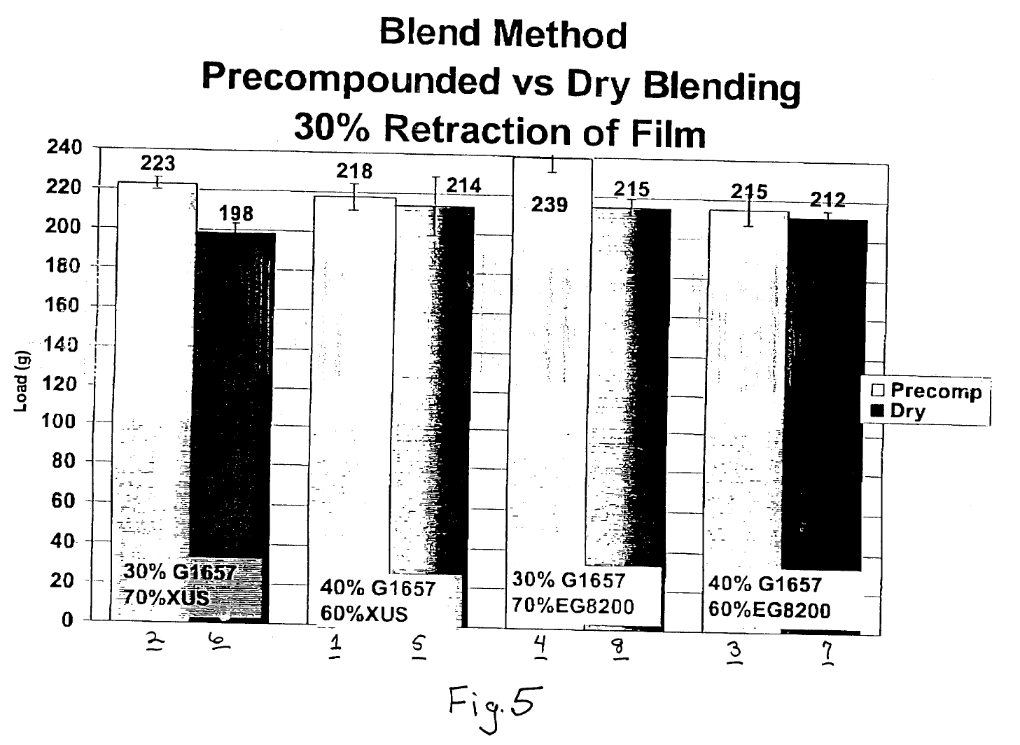

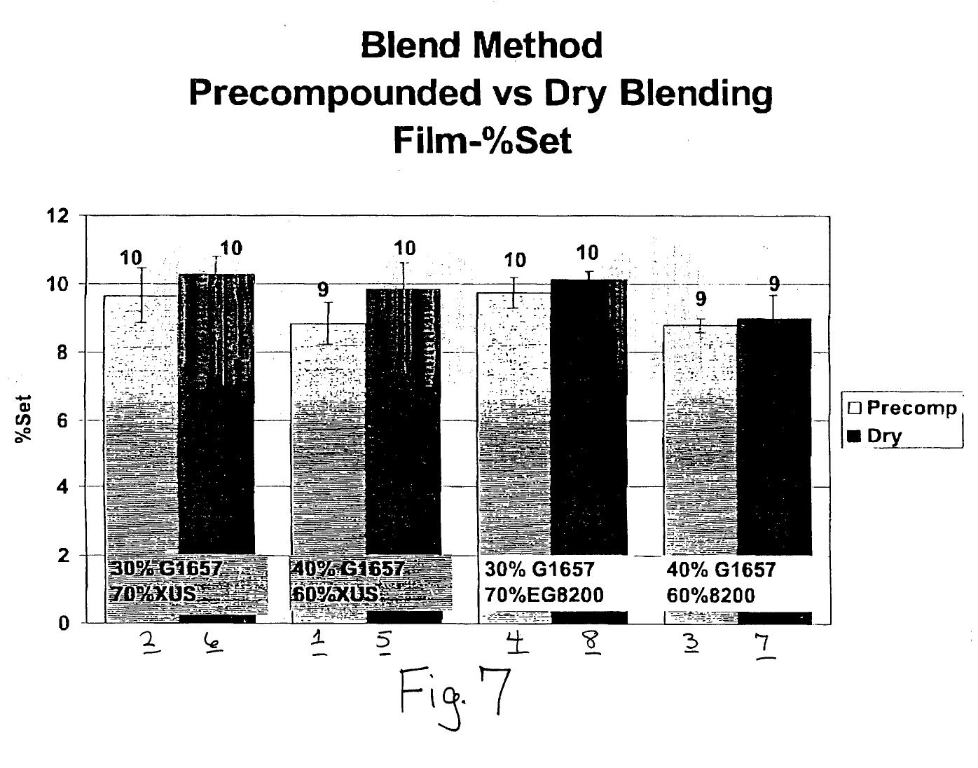

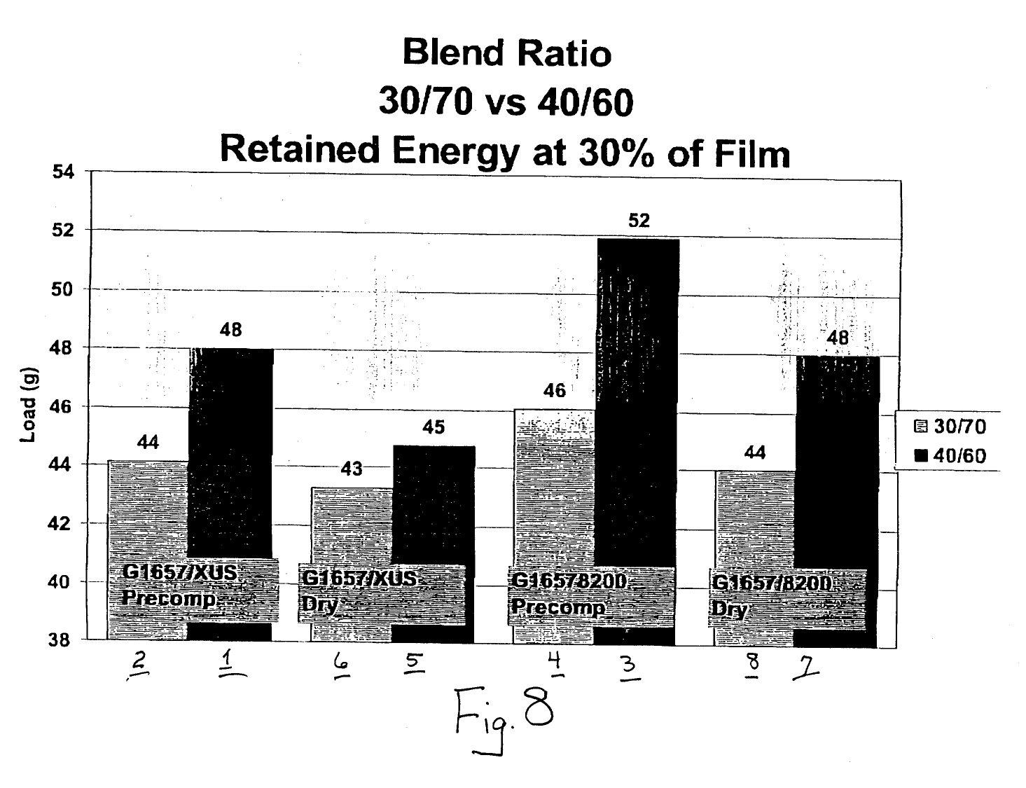

By way of example, to obtain the stress/strain data of FIGS. 4-11, samples of the elastic blend material, or samples of the elastic blend material laminates, can be placed in the clamps of a constant rate of extension (CRE) load frame, such as a SINTECH tensile tester available from Materials Testing Corporation, Minneapolis, Minn., Model No. II. Sample dimensions may desirably be 3 inches wide and 7 inches long, or other dimensions suitable to the test procedure and apparatus.

Again by way of example, starting at a 3 inch gauge length between the sample grips, the test sample may be elongated at 20 in./min. to 100% elongation (6 in. jaw-span). The tester then returns to the original 3 inch gauge length position completing a test cycle. Tension data can then be taken at 30% extension levels of the sample for the extension and retraction half cycles. Hysteresis, and energy retention values may be obtained from the one cycle extension/retraction test. The present examples were tested for one extension/retraction cycle although it is possible to run additional cycles for each sample.

Table I and Table II below compare a 70/30 dry blend and precompounded blend, respectively, made with a single screw, two stage mixer (i.e. including a spin pump for maintaining pressure on the die) and providing a fourteen inch width elastic film. Again it can be seen that at roughly comparable basis weights of the films, no significant differences in the loads at 30% extension or 30% retraction exist between the dry-blend material and precompounded blend material.

TABLE I
70/30 dry blend

| Load at 30% Extension | Load at 30% Retraction | Film Basis Weight |
|---|---|---|
| 631 g | 126 g | 27 gsm |
| 677 g | 150 g | 34 gsm |

TABLE II
70/30 precompounded

| Load at 30% Extension | Load at 30% Retraction | Film Basis Weight |
|---|---|---|
| 609 g | 135 g | 32 gsm |
| 704 g | 180 g | 36 gsm |

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the exemplary embodiments, yet the absence of a particular advantage shall not be construed to necessarily mean that such an embodiment is outside the scope of the present invention.

We claim:

1. An elastic laminate, comprising:
a) a layer of an elastic material having a first and a second side;
b) at least one layer of non-elastic material;
c) the layer of elastic material bonded to the at least one layer of a non-elastic material on at least one of the first and second sides of the elastic material;
d) wherein the elastic material comprises a dry-blend comprising:
i) about 50-80% by weight of a narrow polydispersity number polymer with a density between about 0.80 and 0.95 g/cc; and
ii) about 20-50% by weight of an elastomeric material, and
iii) wherein the blend is essentially free of tackifiers and fillers.

2. The elastic laminate of claim 1 wherein the narrow polydispersity number polymer is a single site catalyzed polyolefin.

3. The elastic laminate of claim 1 wherein the narrow polydispersity number polymer is a metallocene catalyzed polyethylene.

4. The elastic laminate of claim 1 wherein the narrow polydispersity number polymer is a metallocene catalyzed polyethylene having a density of between about 0.86 to about 0.90 g/cc.

5. The elastic laminate of claim 1 wherein the narrow polydispersity number polymer is a metallocene catalyzed polyethylene having a density of less than 0.90 g/cc.

6. The elastic laminate of claim 1 wherein the elastomeric material is a styrenic block copolymer.

7. The elastic laminate of claim 2 wherein the elastomeric material is a styrenic block copolymer.

8. The elastic laminate of claim 3 wherein the elastomeric material is a styrenic block copolymer.

9. An elastic laminate, comprising:
a) a layer of an elastic material having a first and a second side;
b) at least one layer of non-elastic material;
c) the layer of elastic material bonded to the at least one layer of a non-elastic material on at least one of the first and second sides of the elastic material;
d) wherein the elastic material comprises a dry-blend consisting essentially of:
i) about 50-80% by weight of a narrow polydispersity number polymer with density between about 0.83 and about 0.92 g/cc; and
ii) about 20-50% by weight of an elastomeric material.

10. The elastic laminate of claim 9 wherein the narrow polydispersity number polymer is a single site catalyzed polyolefin.

11. The elastic laminate of claim 9 wherein the narrow polydispersity number polymer is a metallocene catalyzed polyethylene.

12. The elastic laminate of claim 9 wherein the narrow polydispersity number polymer is a metallocene catalyzed polyethylene having a density of between about 0.86 to about 0.90 g/cc.

13. The elastic laminate of claim 9 wherein the narrow polydispersity number polymer is a metallocene catalyzed polyethylene having a density of less than 0.90 g/cc.

14. The elastic laminate of claim 9 wherein the elastomeric material is a styrenic block copolymer.

15. The elastic laminate of claim 10 wherein the elastomeric material is a styrenic block copolymer.

16. The elastic laminate of claim 11 wherein the elastomeric material is a styrenic block copolymer.

17. An elastic laminate, comprising:
a) a layer of an elastic film having a first and a second side;
b) at least one layer of non-elastic material;
c) the layer of elastic film bonded to the at least one layer of a non-elastic material on at least one of the first and second sides of the elastic material;
d) wherein the elastic film consists essentially of a dry-blend consisting of:
i) about 50-80% by weight of a polymer with a polydispersity number of 4 or less and with a density of between about 0.83 and about 0.92 g/cc; and
ii) about 20-50% by weight of an elastomeric material.

18. The elastic laminate of claim 17 wherein the narrow polydispersity number polymer is a single site catalyzed polyolefin.

19. The elastic laminate of claim 17 wherein the narrow polydispersity number polymer is a metallocene catalyzed polyethylene.

20. The elastic laminate of claim 17 wherein the narrow polydispersity number polymer is a metallocene catalyzed polyethylene having a density of between about 0.86 to about 0.90 g/cc.

21. The elastic laminate of claim 17 wherein the narrow polydispersity number polymer is a metallocene catalyzed polyethylene having a density of less than 0.90 g/cc.

22. The elastic laminate of claim 17 wherein the elastomeric material is a styrenic block copolymer.

23. The elastic laminate of claim 18 wherein the elastomeric material is a styrenic block copolymer.

24. The elastic laminate of claim 19 wherein the elastomeric material is a styrenic block copolymer.

25. An absorbent garment, comprising:
I) a liner;
II) an outer cover;
III) an absorbent layer between the liner and the outer cover; and
IV) side panels comprising an elastic laminate having:
a) a layer of an elastic material having a first and a second side;
b) at least one layer of non-elastic material;
c) the layer of elastic material bonded to the at least one layer of a non-elastic material on at least one of the first and second sides of the elastic material;
d) wherein the elastic material comprises a dry-blend comprising:
i) about 50-80% by weight of a narrow polydispersity number polymer with a density between about 0.80 and 0.95 g/cc; and
ii) about 20-50% by weight of an elastomeric material, and
iii) wherein the blend is essentially free of tackifiers and fillers.

26. A method of making an elastic laminate, comprising:
a) making a dry-blend composition of polymers comprising:
i) about 50-80% by weight of a narrow polydispersity number polymer with a density between about 0.80 and 0.95 g/cc; and
ii) about 20-50% by weight of an elastomeric material, and
iii) wherein the blend is essentially free of tackifiers and fillers;
b) forming the dry-blend composition into a layer of an elastic material having a first and a second side;
c) providing at least one layer of non-elastic material;
d) bonding the layer of elastic material to the at least one layer of non-elastic material on at least one of the first and second sides of the elastic material.

27. The method of making an elastic laminate according to claim 26 wherein the narrow polydispersity number polymer is a single site catalyzed polyolefin.

28. The method of making an elastic laminate of claim 26 wherein the narrow polydispersity number polymer is a metallocene catalyzed polyethylene.

29. The method of making an elastic laminate of claim 26 wherein the narrow polydispersity number polymer is a metallocene catalyzed polyethylene having a density of less than 0.90 g/cc.

30. The method of making an elastic laminate of claim 26 wherein the elastomeric material is a styrenic block copolymer.

31. The method of making an elastic laminate of claim 27 wherein the elastomeric material is a styrenic block copolymer.

32. The method of making an elastic laminate of claim 28 wherein the elastomeric material is a styrenic block copolymer.

33. A method of making an elastic laminate, comprising:
a) making a dry-blend composition of polymers consisting essentially of:
i) about 50-80% by weight of a narrow polydispersity number polymer with a density between about 0.80 and 0.95 g/cc; and
ii) about 20-50% by weight of an elastomeric material, and
iii) wherein the blend is essentially free of tackifiers and fillers;
b) forming the dry-blend composition into a layer of an elastic material having a first and a second side;
c) providing at least one layer of non-elastic material;
d) bonding the layer of elastic material to the at least one layer of non-elastic material on at least one of the first and second sides of the elastic material.

34. The method of making an elastic laminate according to claim 33 wherein the narrow polydispersity number polymer is a single site catalyzed polyolefin.

35. The method of making an elastic laminate of claim 33 wherein the narrow polydispersity number polymer is a metallocene catalyzed polyethylene.

36. The method of making an elastic laminate of claim 33 wherein the narrow polydispersity number polymer is a metallocene catalyzed polyethylene having a density of less than 0.90 g/cc.

37. The method of making an elastic laminate of claim 34 wherein the elastomeric material is a styrenic block copolymer.

38. The method of making an elastic laminate of claim 35 wherein the elastomeric material is a styrenic block copolymer.

39. The method of making an elastic laminate of claim 36 wherein the elastomeric material is a styrenic block copolymer.
